(19) United States
(12) Patent Application Publication
(10) Pub. No.: US 2025/0281472 A1
(43) Pub. Date: Sep. 11, 2025

(54) OPHTHALMIC COMPOSITION INHIBITING OCCURRENCE OF N-OXOPYRIDINE COMPOUND FOR PREVENTING OR TREATING EYE DISEASE

(71) Applicants: Samjin Pharmaceutical Co., Ltd., Seoul (KR); AptaBio Therapeutics Inc., Yongin-si (KR)

(72) Inventors: Min-hyo Ki, Seongnam-si (KR); Jin-cheul Kim, Seoul (KR); Mee-Hwa Choi, Yongin-si (KR); Dong-Ha Lee, Suwon-si (KR); Sung Hwan Moon, Suwon-si (KR); Soo Jin Lee, Suwon-si (KR)

(73) Assignees: Samjin Pharmaceutical Co., Ltd., Seoul (KR); AptaBio Therapeutics Inc., Yongin-si (KR)

(21) Appl. No.: 18/552,011

(22) PCT Filed: Mar. 23, 2022
(86) PCT No.: PCT/IB2022/052631

§ 371 Date: Sep. 22, 2023

(30) Foreign Application Priority Data
Mar. 24, 2021 (KR) 10-2021-0038356

(51) Int. Cl. A61K 31/4439; A61K 9/00; A61K 9/08; A61K 47/10; A61K 47/20; A61K 47/26; A61K 47/40; A61K 47/44; A61P 27/02

(52) U.S. Cl. CPC A61K 31/4439; A61K 9/0048; A61K 9/08; A61K 47/10; A61K 47/20; A61K 47/26; A61K 47/40; A61K 47/44; A61P 27/02

(57) ABSTRACT

The present invention relates to an eye drop preparation which contains an active ingredient selected from 3-phenyl-4-propyl-1-(pyridin-2-yl)-1H-pyrazol-5-ol compound or pharmaceutically acceptable salts thereof, an antioxidant substance and thus N-oxopyridine compound in an amount of less than about 3%. The eye drop according to the present invention is excellent in stability and safety and shows an excellent effect on prevention or treatment of eye diseases including posterior segment eye diseases simply through instillation rather than a direct injection into an eyeball.

### TECHNICAL FIELD

The present invention relates to an eye drop preparation containing an active ingredient selected from 3-phenyl-4-propyl-1-(pyridin-2-yl)-1H-pyrazol-5-ol or pharmaceutically acceptable salts thereof, which is a composition for minimizing the occurrence of N-oxopyridine compound during a long-term storage. The eye drop composition according to the present invention is excellent in storage stability and safety and shows an excellent effect on prevention or treatment of eye diseases in such a way that an active ingredient thereof reaches not only an anterior segment of an eyeball but also a posterior segment of the eyeball simply through instillation rather than a direct injection into the eyeball for the prevention or treatment of eye diseases.

### BACKGROUND

An eye disease intended by the present invention refers to a disease which affects an eye or a part of the eye or a specific area of the eye or is associated with a specific area of the eye. The anterior segment eye disease refers to a disease which occurs to conjunctiva, cornea, anterior chamber of eye, iris, posterior chamber of eye, crystalline lens or lens capsule, and anterior area. And, the posterior segment eye disease refers to a disease which occurs to choroid or a sclera, a vitreous humor, a vitreous chamber, a retina, optic nerves, and blood vessels and nerves passing a posterior region or area.

In terms of formulations applicable to eye diseases, the eyeball is composed of a variety of complex tissues, and thus has a limit to the delivery of pharmacologically active ingredients into the eye tissues through instillation. For this reason, besides a therapeutic agent for diseases associated with cornea and conjunctiva, there is currently no product used in the form of eye drops for eye diseases including the posterior segment, and all the products are administered through injection administration methods such as intravitreal injection, periocular injection, transscleral injection, etc.

Medicines such as Eylea and Lucentis, which have been recently developed to treat retina & posterior segment diseases, may be also administered only through an intraocular injection, thus making it difficult to be frequently administered, and may show a patient's poor compliance to medication due to repeated injections, as well as accompanying side effects such as bleeding, pain, infection, inflammation, retinal detachment, etc. and there is even a risk of blindness due to injections in severe cases. In addition, due to the above problems, the patient's compliance to medication is poor and it is difficult to continue treatment for five years or more due to severe rejection of direct injection into the eye tissues, and thus there is a need for developing an eye drop formulation for the treatment of posterior segment diseases.

Accordingly, the present invention is to provide a novel eye drop containing 3-phenyl-4-propyl-1-(pyridin-2-yl)-1H-pyrazol-5-ol, which has the compliance to medication for the treatment of the above eyeball diseases and has an excellent therapeutic effect on posterior segment eye diseases by resolving the patient's rejection.

As the prior art related thereto, Patent Document 1 describes a composition for preventing and treating osteoporosis, and Patent Document 2 describes a composition for preventing or treating kidney disease, in which a pyrazole-based compound of the present invention is specified as an active ingredient. However, there is no mention of using the pyrazole-based compound (3-phenyl-4-propyl-1-(pyridin-2-yl)-1H-pyrazol-5-ol) as an eye drop and there is no example of using an eye drop for the treatment of osteoporosis and kidney disease, which is specified in prior documents. And Patent Document 3 describes a use of the pyrazole-based compound for the prevention and treatment of eye diseases, in which all experimental examples and examples were tested by a direct intraocular injection without any description about the composition of eye drops. Furthermore, it has not been confirmed that an amount of the N-oxopyridine compound is increased a lot when 3-phenyl-4-propyl-1-(pyridin-2-yl)-1H-pyrazol-5-ol found in the present invention is stored in a liquid form for six months or more, and thus there is a difference from the eye drop composition of the present invention, which proposes a method for solving the above problem.

### RELATED ART REFERENCES

### Patent Documents

(Patent Document 1) Korean Registered Patent No. 10-1280160
(Patent Document 2) Korean Registered Patent No. 10-1633957
(Patent Document 3) Korean Registered Patent No. 10-1821593

### DETAILED DESCRIPTION OF THE INVENTION

### Technical Problem

One object of the present invention is to provide an eye drop of 3-phenyl-4-propyl-1-(pyridin-2-yl)-1H-pyrazol-5-ol compound showing a valid effect on treatment of eye diseases with excellent long-term storage stability.

In addition, one object of the present invention is to provide an eye drop showing not only a high patient's compliance with medication but also excellent storage stability and safety by containing at least one antioxidant substance, and thus may be useful as a therapeutic agent for eye diseases.

Furthermore, one object of the present invention is to provide a novel therapeutic option, which shows an excellent patient's compliance, to patients even with posterior segment eye diseases such as macular degeneration by allowing a composition with secured long-term stability and safety to be delivered simply through eye drop administration without any injection into the eyeball, so as to show an effect similar to or more excellent than an anti-VEGF drug in the existing form of intraocular injection.

### Technical Solution

A method for achieving the objects of the present invention is as follows. All combinations of various elements disclosed in the present invention belong to the scope of the present invention, but it cannot be considered that the scope of the present invention is limited by the specific description described below.

The present invention may provide an eye drop for preventing or treating eye diseases, which contains an active ingredient selected from 3-phenyl-4-propyl-1-(pyridin-2-yl)-1H-pyrazol-5-ol represented by formula 1 below or pharmaceutically acceptable salts thereof, and an N-oxopyridine compound represented by formula 2 below in an amount of less than about 3%.

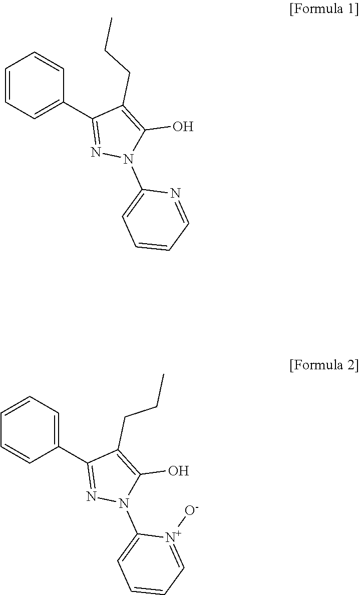 [SMILES: CCCc1c(-c2ccccc2)nn(-c2ccccn2)c1O ; CCCc1c(-c2ccccc2)nn(-c2cccc[n+]2[O-])c1O]

The eye drop according to the present invention may maintain excellent stability by minimizing the occurrence of the N-oxopyridine compound, which may rapidly increase during a long period of use.

The eye drop for preventing or treating eye diseases may further include an antioxidant substance.

According to one embodiment, the present invention may provide an eye drop for preventing or treating eye diseases, which contains 3-phenyl-4-propyl-1-(pyridin-2-yl)-1H-pyrazol-5-ol represented by formula 1 below or pharmaceutically acceptable salts thereof as an active ingredient, and contains at least one antioxidant substance selected from cysteine, N-acetyl cysteine and monothioglycerol.

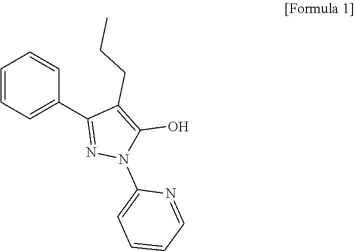 [SMILES: CCCc1c(-c2ccccc2)nn(-c2ccccn2)c1O]

According to one embodiment, the present invention may provide an eye drop for preventing or treating eye diseases, which contains 3-phenyl-4-propyl-1-(pyridin-2-yl)-1H-pyrazol-5-ol represented by formula 1 below or pharmaceutically acceptable salts thereof as an active ingredient, and contains monothioglycerol as an antioxidant substance.

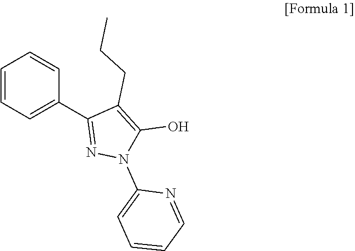 [SMILES: CCCc1c(-c2ccccc2)nn(-c2ccccn2)c1O]

In the present invention, the pharmaceutically acceptable salt of 3-phenyl-4-propyl-1-(pyridin-2-yl)-1H-pyrazol-5-ol described above may be hydrochloride.

In the present invention, the above-mentioned eye drop may contain an antioxidant substance in an amount of about 0.1 wt % to about 5.0 wt % based on the total weight of the eye drop.

In the present invention, the above-mentioned eye drop may contain the N-oxopyridine compound represented by formula 2 below in an amount of less than about 3%.

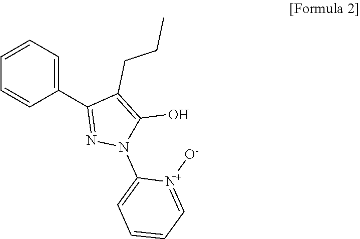 [SMILES: CCCc1c(-c2ccccc2)nn(-c2cccc[n+]2[O-])c1O]

The eye drop may contain the N-oxopyridine compound represented by above formula 2 in an amount of less than about 3%, even after storage for about six months under the condition of about 25° C. temperature and about 60% relative humidity.

In the present invention, the eye disease may be the posterior segment eye disease. The posterior segment eye disease may be at least one selected from the group consisting of diabetic retinopathy (DR), diabetic macular edema, age-related macular degeneration, acute macular neuroretinopathy, premature retinopathy (ROP), polypoidal choroidal vasculopathy, ischemic proliferative retinopathy, retinitis pigmentosa, cone dystrophy, Behcet's disease, retinal disorders, proliferative vitreoretinopathy (PVR), retinal arterial obstruction, retinal venous obstruction, retinitis, uveitis, Lever's optic nerve atrophy, retinal detachment, retinal pigment epithelium detachment, neovascular glaucoma, retinal neovascularization and choroidal neovascularization (CNV), trauma of the posterior segment, radiation retinopathy, epiretinal membrane disorder, branch retinal vein occlusion, anterior ischemic optic nerve disorder, non-retinopathy diabetic retinal dysfunction, and glaucoma.

When the eye drop according to the present invention is stored in a liquid form for about six months or more, there is a very unusual problem in which 3-phenyl-4-propyl-1-(pyridin-2-yl)-1H-pyrazol-5-ol, which is an active ingredient of the present invention, is converted a lot into the N-oxopyridine compound, which is an ineffective by-product, in an amount of 10 to 30%. In a preclinical toxicity test conducted to confirm the safety of the composition of the present invention, the test was conducted with a composition controlled so that the amount of N-oxopyridine compound does not exceed about 3%, and when the amount of N-oxopyridine compound exceeds about 3%, a problem about safety may occur. And, if a large amount of impurities continuously increase over time, it may be also difficult to accept this increase in terms of stability and quality control of general medicines. Accordingly, it is impossible to use a composition as a drug medicine in terms of stability and safety, if the composition contains more than about 3% of N-oxopyridine compound, which is a by-product of 3-phenyl-4-propyl-1-(pyridin-2-yl)-1H-pyrazol-5-ol, which is an active ingredient of the present invention. Thus, there is an essential need to develop a composition which minimizes the N-oxopyridine compound so that the eye drop of the present invention may be developed as a drug medicine. In particular, considering that eye drops need to be stored in a liquid form for a long period of time due to the properties thereof, there is a need to develop a novel composition that minimizes the N-oxopyridine compound in a liquid form.

It was confirmed that the novel eye drop composition of the present invention effectively inhibits the production of the N-oxopyridine compound, which is an impurity, thereby enabling the development of eye drops with excellent stability and safety. In addition, it was confirmed through an animal test that the composition of the present invention with secured long-term stability shows an excellent therapeutic effect on the posterior segment simply through instillation, which is equal to or greater than that of the existing therapeutic agents by intraocular injection.

Accordingly, it was confirmed that the composition of the present invention may be used as an eye drop for preventing or treating eye diseases including posterior segment eye disease.
(1) In the present invention, an eye drop for preventing or treating eye diseases comprises the following: 3-phenyl-4-propyl-1-(pyridin-2-yl)-1H-pyrazol-5-ol represented by formula 1 below or pharmaceutically acceptable salts thereof as an active ingredient; N-oxopyridine compound represented by formula 2 below in an amount of less than about 3%.

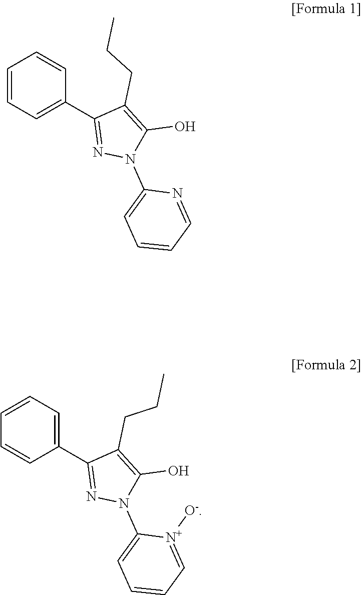

(2) The eye drop according to (1) above further comprises an antioxidant substance.
(3) In the present invention, an eye drop for preventing or treating eye diseases comprises the following: 3-phenyl-4-propyl-1-(pyridin-2-yl)-1H-pyrazol-5-ol represented by formula 1 below or pharmaceutically acceptable salts thereof as an active ingredient; at least one antioxidant substance selected from cysteine, N-acetyl cysteine and monothioglycerol.

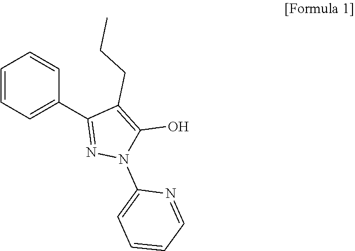 [SMILES: CCCc1c(-c2ccccc2)nn(-c2ccccn2)c1O]

(4) In the present invention, an eye drop for preventing or treating eye diseases comprises the following: 3-phenyl-4-propyl-1-(pyridin-2-yl)-1H-pyrazol-5-ol represented by formula 1 below or pharmaceutically acceptable salts thereof as an active ingredient; monothioglycerol as an antioxidant substance.

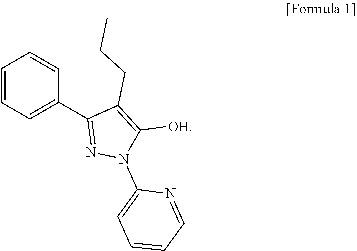 [SMILES: CCCc1c(-c2ccccc2)nn(-c2ccccn2)c1O]

(5) In (1), (2), (3) or (4) above, the pharmaceutically acceptable salt is hydrochloride.
(6) The eye drop according to (2), (3), (4) or (5) above comprises the antioxidant substance in an amount of about 0.1 wt % to about 5.0 wt % based on the total weight of the eye drop.
(7) The eye drop according to (3), (4), (5) or (6) above comprises N-oxopyridine compound represented by formula 2 below in an amount of less than about 3%.

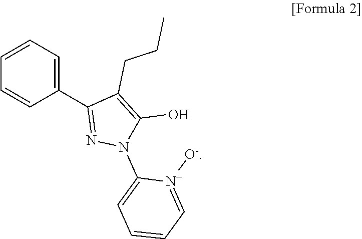

(8) The eye drop according to (2), (3), (4), (5), (6) or (7) above comprises the N-oxopyridine compound represented by formula 2 above in an amount of less than about 3%, even after storage for about six months under the condition of about 25° C. temperature and about 60% relative humidity.
(9) In (1), (2), (3), (4), (5), (6), (7) or (8) above, the eye disease is a posterior segment eye disease.
(10) In (1), (2), (3), (4), (5), (6), (7), (8) or (9) above, the posterior segment eye disease is at least one selected from the group consisting of diabetic retinopathy (DR), diabetic macular edema, age-related macular degeneration, acute macular neuroretinopathy, retinopathy of prematurity (ROP), polypoidal choroidal vasculopathy, ischemic proliferative retinopathy, retinitis pigmentosa, cone dystrophy, Behcet's disease, retinal disorders, proliferative vitreoretinopathy (PVR), retinal artery occlusion, retinal vein occlusion, retinitis, uveitis, Leber's hereditary optic neuropathy, retinal detachment, retinal pigment epithelial detachment, neovascular glaucoma, retinal neovascularization and choroidal neovascularization (CNV), trauma of the posterior segment, radiation retinopathy, epiretinal membrane disorder, branch retinal vein occlusion, anterior ischemic optic nerve disorder, non-retinopathy diabetic retinal dysfunction, and glaucoma.
(11) In the present invention, a method for preventing or treating eye diseases comprises administering the eye drop according to (1), (2), (3), (4), (5), (6), (7), (8), (9) or (10) above to a subject in need thereof.
(12) In the present invention, the use of the eye drop according to (1), (2), (3), (4), (5), (6), (7), (8), (9) or (10) above is for preventing or treating eye diseases.
(13) In the present invention, the use of the eye drop according to (1), (2), (3), (4), (5), (6), (7), (8), (9) or (10) above is in the manufacture of a medicament for preventing or treating eye diseases.

Hereinafter, in the present specification, the “active ingredient” may refer to “3-phenyl-4-propyl-1-(pyridin-2-yl)-1H-pyrazol-5-ol or pharmaceutically acceptable salts thereof,” unless otherwise specified.

In the present invention, pharmaceutically acceptable salts may refer to the salts conventionally used in a pharmaceutical industry, for example, inorganic ion salts prepared from calcium, potassium, sodium, magnesium and the like; inorganic acid salts prepared from hydrochloric acid, nitric acid, phosphoric acid, bromic acid, iodic acid, perchloric acid, tartaric acid, sulfuric acid and the like; organic acid salts prepared from acetic acid, trifluoroacetic acid, citric acid, maleic acid, succinic acid, oxalic acid, benzoic acid, fumaric acid, mandelic acid, propionic acid, lactic acid, glycolic acid, gluconic acid, galacturonic acid, glutamic acid, glutaric acid, glucuronic acid, aspartic acid, ascorbic acid, carbonic acid, vanillic acid, hydroiodic acid and the like; sulfonic acid salts prepared from methanesulfonic acid, ethanesulfonic acid, benzenesulfonic acid, p-toluenesulfonic acid, naphthalenesulfonic acid and the like; amino acid salts prepared from glycine, arginine, lysine and the like; amine salts prepared from trimethylamine, triethylamine, ammonia, pyridine, picoline and the like; but types of salts meant in the present invention are not limited to those listed salts. For example, in one embodiment, the pharmaceutically acceptable salt may be hydrochloride.

The present invention is to provide a composition capable of providing an eye drop treatment for various eye diseases. For this purpose, the composition has the essential condition of securing the stability of the active ingredient in a liquid state as an eye drop for about six months or more. When 3-phenyl-4-propyl-1-(pyridin-2-yl)-1H-pyrazol-5-ol, the active ingredient of the present invention, is prepared as a liquid eye drop and stored for about six months or more under the condition of a long-term storage test, it was confirmed that a specific impurity is unusually and highly increased. And as a result of confirming a structure of the corresponding impurity, it was confirmed that the impurity is 3-phenyl-4-propyl-1-(N-oxopyridin-2-yl)-1H-pyrazol-5-ol (hereinafter, N-oxopyridine compound), in which an N-oxide structure is formed in a pyridine structure of the active ingredient. If the pH condition is adjusted to a neutral region where the active ingredient is stable, most impurities are controlled to be within about 0.5%. However, it was confirmed that the N-oxopyridine compound drastically increases to a level exceeding about 30% with a decrease in the active ingredient content even when the pH is controlled.

The N-oxopyridine compound is identified as a substance produced when a pyridine structure inside the active ingredient reacts with water used in the composition of the eye drop or oxygen present in the composition due to the structural properties of 3-phenyl-4-propyl-1-(pyridin-2-yl)-1H-pyrazol-5-ol, which is the active ingredient of the present invention. In case of 3-phenyl-4-propyl-1-(pyridin-2-yl)-1H-pyrazol-5-ol of the present invention, a pyridine structure was converted into the N-oxopyridine compound at a very high rate during a long-term storage in a liquid form compared to other compounds having a general pyridine structure. Thus, it is difficult to develop a drug medicine only by solubilizing the active ingredient of the present invention and preparing the same as an eye drop and thus there is a need to develop a composition capable of minimizing the occurrence of the N-oxopyridine compound. In particular, considering that the range of impurities, including the N-oxopyridine compound with safety confirmed through a preclinical toxicity test of the active ingredient of the present invention, is less than about 3%, there is an essential need to develop a composition for minimizing the corresponding impurities to less than about 3% to ensure safety.

If an excessive amount of impurities without safety secured are produced in a liquid formulation such as eye drops, the effectiveness of the active ingredient may decrease along with the occurrence of ill-defined side effects, and thus it is very important to set standards for the corresponding impurities. In this regard, an antioxidant substance was added to the composition of the present invention as a method for minimizing the production of the N-oxopyridine compound in order to ensure safety and stability during the period of use of the eye drop of the present invention, and it was confirmed that the eye drop containing the antioxidant substance used in one example of the present invention makes an excellent improvement in stability as a drug during a storage period compared to the eye drops not containing the corresponding compound. Moreover, it was confirmed that the antioxidant substance used in one embodiment has a very excellent effect on stabilizing the active ingredient of the present invention.

The antioxidant substance used in the present invention may serve to prevent “3-phenyl-4-propyl-1-(pyridin-2-yl)-1H-pyrazol-5-ol,” which is the active ingredient of the present invention, from being converted into “3-phenyl-4-propyl-1-(N-oxo pyridin-2-yl)-1H-pyrazol-5-ol (N-oxopyridine compound).” An antioxidant substance usable in the present invention may be at least one compound selected from cysteine, N-acetylcysteine, homocysteine, glutathione, dithioerythritol (1,4-dithioerythritol) and monothioglycerol (thioglycerol), but is not limited thereto. In the present invention, the antioxidant substance may be included in an amount of about 0.1 to about 5.0 (w/v) % based on the total weight of the composition, so as to prevent the active ingredient from being converted into the N-oxopyridine compound, thereby ensuring long-term stability and safety of the drug medicine.

In addition, the eye drop of the present invention may further contain at least one additive selected from solubilizers. In the present invention, the “solubilizer” may be a pharmaceutical additive used to increase the solubility of a drug in an aqueous solvent, and may be selected from the group consisting of solubilizing aids, surfactants, clathrates, and mixtures thereof. The “solubilizing aid” may be an additive that increases the solubility of an active ingredient in a liquid form, and may be selected from conventional additives used as a solvent in the pharmaceutical industry. The “surfactant” may be a substance used to lower an interfacial tension when an active ingredient is dissolved in water or aqueous solution, and may be selected from the group consisting of ionic surfactants, nonionic surfactants, polymeric surfactants, and mixtures thereof. The “clathrate” may be an additive that increases solubility through clathration of the active ingredient, and may be selected from the group consisting of cyclodextrin, cyclodextrin derivatives, and mixtures thereof.

When the eye drop of one embodiment contains a solubilizer, the solubilizing aid may include ethanol, glycerin, propylene glycol, castor oil, medium chain fatty acids (capric and caprylic) mono-diglycerin and mixtures thereof; the surfactant may include polyoxyl castor oil, polyoxyethylene sorbitan fatty acid esters, polyoxyethylene fatty acids, polyoxyethylene-polyoxypropylene copolymers and mixtures thereof; the clathrate may include cyclodextrin, cyclodextrin substituted with hydroxyalkyl group, cyclodextrin substituted with alkyl ether, 2-hydroxypropyl-β-cyclodextrin, sulfobutyl ether-β-cyclodextrin, and mixtures thereof. Thus, it is possible to include the additives used in conventional techniques for preparing eye drops, but is not limited thereto.

In addition, the eye drop of the present invention may further contain at least one additive selected from the group consisting of a thickener and a preservative. When the eye drop of one embodiment includes a thickener and a preservative, the type is not particularly limited, and the thickener may include polyvinyl alcohol, polyvinylpyrrolidone, hydroxypropylmethylcellulose, hydroxypropylcellulose, and mixtures thereof; and the preservative may include benzalkonium chloride, chlorobutanol, methyl paraoxybenzoate, propyl paraoxybenzoate, and mixtures thereof. Thus, it is possible to use the thickeners and preservatives used in conventional techniques for preparing eye drops.

The eye diseases intended by the present invention may include anterior segment eye diseases and posterior segment eye diseases, and preferably the posterior segment eye diseases may be selected. Anterior segment eye diseases may refer to diseases which affect or involve an anterior area and region such as muscles around the eye, eyelids, or eyeball tissues or body fluids, which are located on a posterior wall of a lens capsule or in front of a ciliary muscle. The anterior segment eye diseases may refer to at least one selected from the group consisting of dry eye, keratitis, conjunctivitis, scleritis, cataract, pinguecula, conjunctival nevus, ota nevus and corneal opacity (corneal tattooing), or at least one selected from the group consisting of other diseases associated with conjunctiva, cornea, anterior chamber of eye, iris, posterior chamber of eye, crystalline lens or lens capsule, and blood vessels and nerves passing the anterior area and region.

Posterior segment eye diseases may refer to at least one selected from the group consisting of diabetic retinopathy (DR), diabetic macular edema, age-related macular degeneration, acute macular neuroretinopathy, premature retinopathy (ROP), polypoidal choroidal vasculopathy, ischemic proliferative retinopathy, retinitis pigmentosa, cone dystrophy, Behcet's disease, retinal disorders, proliferative vitreoretinopathy (PVR), retinal arterial obstruction, retinal venous obstruction, retinitis, uveitis, Lever's optic nerve atrophy, retinal detachment, retinal pigment epithelium detachment, neovascular glaucoma, retinal neovascularization and choroidal neovascularization (CNV), trauma of the posterior segment, posterior segment eye diseases caused or affected by ocular laser treatment, posterior segment eye diseases caused or affected by photodynamic therapy or photocoagulation, radiation retinopathy, epiretinal membrane disorder, branch retinal vein occlusion, anterior ischemic optic nerve disorder, non-retinopathy diabetic retinal dysfunction, glaucoma, and diseases caused or affected by glaucoma.

In fact, as a result of conducting an efficacy test of the eye drop composition of the present invention with secured long-term stability by using CNV-induced animals used for the development of a therapeutic agent for macular degeneration and macular edema, which are typical posterior segment eye diseases, it was confirmed that the composition has an excellent therapeutic effect simply through instillation.

In the case of posterior segment eye diseases accompanied by blindness, such as macular degeneration and macular edema, a direct injection administration into eye ball tissues such as an intraocular injection has been used as the only treatment method. However, this intraocular injection fails to be periodically performed since the third year of treatment due to a patient's rejection of intraocular injection. Thus, it is known that such failure of intraocular injection eventually leads to gradual blindness. In the case of applying the eye drop composition of the present invention with secured stability and safety for these diseases, there is an advantage of addressing various side effects such as fear, pain, bleeding, inflammation, blindness and the like caused by the injection of the conventional therapeutic agent for posterior segment eye diseases, which needs to be directly injected into the eyeball. In addition, the eye drop according to one embodiment has secured advantages such as convenience, etc., in that the eye drop may be easily administered by the patients themselves, and thus this eye drop may be very useful for patients with eye diseases including posterior segment eye diseases.

According to a preferred embodiment aspect of the present invention, the composition of the present invention may provide an eye drop which has secured long-term stability and safety of the 3-phenyl-4-propyl-1-(pyridin-2-yl)-1H-pyrazol-5-ol compound, including an antioxidant substance, and may be used to treat not only anterior segment eye diseases but also posterior segment eye diseases simply through instillation without any injection.

The eye drop composition may be prepared as a liquid eye drop, but embodiments are not limited thereto, and may be prepared into a dosage form of an ointment, a gelling agent, etc., according to a formulation method.

The present invention may provide a method for preventing or treating eye diseases including administering the eye drop which contains 3-phenyl-4-propyl-1-(pyridin-2-yl)-1H-pyrazol-5-ol represented by formula 1 or pharmaceutically acceptable salts thereof as an active ingredient, and the N-oxopyridine compound represented by formula 2 in an amount of less than about 3% to a subject in need thereof.

The present invention may provide a method for preventing or treating eye diseases including administering the eye drop which contains 3-phenyl-4-propyl-1-(pyridin-2-yl)-1H-pyrazol-5-ol represented by formula 1 or pharmaceutically acceptable salts thereof as an active ingredient, at least one antioxidant substance, and the N-oxopyridine compound in an amount of less than about 3% to a subject in need of treatment.

The present invention may provide a use of the eye drop which contains 3-phenyl-4-propyl-1-(pyridin-2-yl)-1H-pyrazol-5-ol represented by formula 1 or pharmaceutically acceptable salts thereof as an active ingredient, and the N-oxopyridine compound represented by formula 2 in an amount of less than about 3% for preventing or treating eye diseases.

The present invention may provide a use of the eye drop which contains 3-phenyl-4-propyl-1-(pyridin-2-yl)-1H-pyrazol-5-ol represented by formula 1 or pharmaceutically acceptable salts thereof as an active ingredient, at least one antioxidant substance, and the N-oxopyridine compound in an amount of less than about 3% for preventing or treating eye diseases.

The present invention may provide a use of an ophthalmic composition containing 3-phenyl-4-propyl-1-(pyridin-2-yl)-1H-pyrazol-5-ol represented by formula 1 or a pharmaceutically acceptable salt thereof as an active ingredient, and N-oxopyridine compound represented by formula 2 in an amount of less than about 3%, in preparation of an eye drop for preventing or treating eye diseases.

The present invention may provide a method for preparing the eye drop, including a step of mixing 3-phenyl-4-propyl-1-(pyridin-2-yl)-1H-pyrazol-5-ol represented by formula 1 or pharmaceutically acceptable salts thereof, and an antioxidant substance. The above preparation method may further include a step of mixing other additives.

In the present specification, “eye drop” and “ophthalmic composition” may be used interchangeably.

For the use of the eye drop according to the present invention, the use of the eye drop composition, the preparation method thereof, and the preventive or therapeutic method including the step of administering the eye drop, the description of the eye drop mentioned above may be equally applied if not contradictory to each other.

### Advantageous Effects

The present invention relates to an eye drop of 3-phenyl-4-propyl-1-(pyridin-2-yl)-1H-pyrazol-5-ol compound showing a valid effect on treatment of eye diseases, and the eye drop shows not only a high patient's compliance with medication but also excellent storage stability and safety by including at least one antioxidant substance, and thus can be useful as a therapeutic agent for eye diseases. The present invention is expected to provide a novel therapeutic option, which shows an excellent patient's compliance, to patients even with posterior segment eye diseases such as macular degeneration by allowing a composition with secured long-term stability and safety to be delivered simply through eye drop administration without an injection into the eyeball, so as to show an effect similar to or more excellent than an anti-VEGF drug in the existing form of intraocular injection.

### BRIEF DESCRIPTION OF THE DRAWINGS

FIG. 1 is a graph showing a change in the content of active ingredients measured after a long-term stability test according to Test Example 1.

FIG. 2 is a graph showing an amount of the N-oxopyridine compound produced measured after a long-term stability test according to Test Example 1.

FIG. 3 is a graph showing the result of observing and digitizing the size of CNV lesions after performing a retinal imaging evaluation using FFA after administration of the composition of the present invention (Example 7) and the control group according to Test Example 2.

### MODE FOR INVENTION

Hereinafter, the present invention will be described in more detail through exemplary embodiments. These exemplary embodiments are provided only for the purpose of illustrating the present invention in more detail, and thus it will be apparent to those skilled in the art that the scope of the present invention is not limited thereto.

### Examples 1 to 6: Preparing APX-115 Eye Drops (Including Cysteine)

A composition containing 3-phenyl-4-propyl-1-(pyridin-2-yl)-1H-pyrazol-5-ol hydrochloride (APX-115) as an active ingredient and cysteine was prepared as shown in table 1 below.

Ethanol, propylene glycol and/or castor oil were mixed and homogenized, after which APX-115 was added and dissolved, and then polyoxyl 35 castor oil or polysorbate 80 was mixed to prepare a mixed solution in which an active ingredient was dissolved. Hydroxypropyl betadex and cysteine, which is an antioxidant substance, were dissolved in a buffer and homogeneously mixed with the mixed solution in which the active ingredient was dissolved, and then filtered through a 0.22 μm sterile filter.

TABLE 1

| | Raw Material Name | 1 | 2 | 3 | 4 | 5 | 6 |
|---|---|---|---|---|---|---|---|
| Main | APX-115 | 0.1 | 0.25 | 0.25 | 0.25 | 0.5 | 0.5 |
| ingredient | | | | | | | |
| Solubilizing | Ethanol | 0.5 | 0.5 | 1.0 | 1.0 | 1.0 | 1.0 |
| additive | Propylene glycol | — | 0.5 | — | 0.5 | — | 0.5 |
| | Castor oil | 0.5 | — | 1.0 | — | 1.0 | — |
| | Polyoxyl 35 | 5.0 | 5.0 | — | — | 5.0 | 5.0 |
| | Castor oil | | | | | | |
| | Polysorbate 80 | — | — | 5.0 | 5.0 | — | — |
| | Hydroxypropyl | — | 2.5 | — | — | 5.0 | 5.0 |
| | betadex | | | | | | |
| Antioxidant | Cysteine | 0.1 | 0.25 | 0.25 | 0.5 | 0.5 | 0.5 |
| substance | | | | | | | |
| | Buffer | Suitable | Suitable | Suitable | Suitable | Suitable | Suitable |
| | | ammount | amount | amount | amount | amount | amount |

Unit: % (w/v)

In the present invention, anhydrous sodium dihydrogen phosphate and anhydrous sodium phosphate were used as the buffer, and the pH was adjusted to pH 6.5 with 0.5 N sodium hydroxide solution, if necessary.

### Examples 7 to 12: Preparing APX-115 Eye Drops (Including Thioglycerol)

A composition containing 3-phenyl-4-propyl-1-(pyridin-2-yl)-1H-pyrazol-5-ol hydrochloride (APX-115) as an active ingredient and thioglycerol was prepared as shown in table 2 below.

Ethanol, propylene glycol, castor oil and thioglycerol, which is an antioxidant substance, were mixed and homogenized, after which APX-115 was added and dissolved, and then polyoxyl 35 castor oil or polysorbate 80 was mixed to prepare a mixed solution in which an active ingredient was dissolved. A mixed solution obtained by homogeneously mixing a mixture in which the active ingredient is dissolved in a buffer (Examples 7 and 8) or a mixed solution obtained by homogeneously mixing a mixture in which the active ingredient is dissolved in a buffer in which hydroxypropyl betadex is dissolved (Examples 9 and 10) was filtered with a 0.22 μm sterile filter.

TABLE 2

| | Raw Material Name | 7 | 8 | 9 | 10 | 11 | 12 |
|---|---|---|---|---|---|---|---|
| Main | APX-115 | 0.1 | 0.25 | 0.25 | 0.5 | 0.5 | 0.5 |
| ingredient | | | | | | | |
| Solubilizing | Ethanol | 0.5 | 1.0 | 1.0 | 1.0 | 1.0 | 1.0 |
| additive | Propylene glycol | — | 0.5 | 0.5 | — | — | 0.5 |
| | Castor oil | 0.5 | 1.0 | 1.0 | 1.0 | 1.0 | — |
| | Polyoxyl 35 | 5.0 | 5.0 | — | — | — | 5.0 |
| | Castor oil | | | | | | |
| | Polysorbate 80 | — | — | 5.0 | 5.0 | 5.0 | — |
| | Hydroxypropyl | 2.5 | — | — | — | 5.0 | 5.0 |
| | betadex | | | | | | |
| Antioxidant | Thioglycerol | 0.1 | 0.5 | 0.5 | 2.5 | 2.5 | 5.0 |
| substance | | | | | | | |
| | Buffer | Suitable | Suitable | Suitable | Suitable | Suitable | Suitable |
| | | amount | amount | amount | amount | amount | amount |

Unit: % (w/v)

In the present invention, anhydrous sodium dihydrogen phosphate and anhydrous sodium phosphate were used as the buffer, and the pH was adjusted to pH 7.2 with 0.5 N sodium hydroxide solution, if necessary.

### Examples 13 to 18: Preparing APX-115 Eye Drops (Including N-Acetyl Cysteine)

A composition containing 3-phenyl-4-propyl-1-(pyridin-2-yl)-1H-pyrazol-5-ol hydrochloride (APX-115) as an active ingredient and N-acetylcysteine was prepared as shown in table 3 below.

Ethanol, propylene glycol and/or castor oil were mixed and homogenized, after which APX-115 was added and dissolved, and then polyoxyl 35 castor oil or polysorbate 80 was mixed to prepare a mixed solution in which an active ingredient was dissolved. Hydroxypropyl betadex and N-acetylcysteine, which is an antioxidant substance, were dissolved in a buffer and homogeneously mixed with the mixed solution in which the active ingredient was dissolved, and then filtered through a 0.22 μm sterile filter.

TABLE 3

| | Raw Material Name | 13 | 14 | 15 | 16 | 17 | 18 |
|---|---|---|---|---|---|---|---|
| Main | APX-115 | 0.1 | 0.25 | 0.5 | 0.5 | 1.0 | 1.0 |
| ingredient | | | | | | | |
| Solubilizing | Ethanol | 0.5 | 1.0 | 1.0 | 1.0 | 1.0 | 1.0 |
| additive | Propylene glycol | — | — | 0.5 | 0.5 | 0.5 | 0.5 |
| | Castor oil | 0.5 | 1.0 | — | 1.0 | — | 1.0 |
| | Polyoxyl 35 | 5.0 | 5.0 | — | 5.0 | 2.5 | 5.0 |
| | Castor oil | | | | | | |
| | Polysorbate 80 | — | — | 5.0 | 2.5 | 5.0 | 2.5 |
| | Hydroxypropyl | — | — | — | 5.0 | 5.0 | 5.0 |
| | betadex | | | | | | |
| Antioxidant | N-acetylcysteine | 0.25 | 0.5 | 0.5 | 1.0 | 1.0 | 1.0 |
| substance | | | | | | | |
| | Buffer | Suitable | Suitable | Suitable | Suitable | Suitable | Suitable |
| | | amount | amount | amount | amount | amount | amount |

Unit: % (w/v)

In the present invention, anhydrous sodium dihydrogen phosphate and anhydrous sodium phosphate were used as the buffer, and the pH was adjusted to pH 7.8 with 0.5 N sodium hydroxide solution, if necessary.

### Comparative Example 1

Ethanol, propylene glycol and/or castor oil were mixed and homogenized, after which APX-115 was added and dissolved, and then polyoxyl 35 castor oil was mixed to prepare a mixed solution in which an active ingredient was dissolved. After homogeneously mixed with the mixed solution in which the active ingredient was dissolved in the buffer, the resulting mixture was filtered through a 0.22 μm sterile filter to prepare a composition (pH 7.2) not containing an antioxidant substance.

### Test Example 1: Stability Test

The solutions prepared in above Examples and Comparative Examples were stored under the condition of about 25° C./about 60% RH. A content of APX-115 and an amount of the N-oxopyridine compound produced by conversion of APX-115 for about six months were measured by the HPLC method under the conditions described below.

### <Analysis Conditions>

Column: Kromasil C18 (4.6×150 mm, 5 μm)
Column temperature: 30° C.
Mobile phase: 20 mM ammonium formate (pH 3.0)/acetonitrile=20/80 (v/v)
UV wavelength: 293 nm
Injection amount: 10 μl

TABLE 4
Stability test result (content)

| | | content (%) | | |
|---|---|---|---|---|
| (Unit: %) | | Initial | 3 months | 6 months |
| Example | 3 (Cysteine) | 98.9 | 96.9 | 96.1 |
| | 4 (Cysteine) | 98.5 | 97.1 | 96.7 |
| | 8 (Thioglycerol) | 101.8 | 102.2 | 101.6 |
| | 10 (Thioglycerol) | 99.5 | 98.9 | 99.1 |
| | 16 (N-acetylcysteine) | 99.9 | 98.7 | 96.8 |
| Comparative | 1 (None) | 100.7 | 86.5 | <70 |
| Example | | | | |

TABLE 5
Stability test result (compound of formula 2)

| | | N-oxopyridine compound (%) | | |
|---|---|---|---|---|
| (Unit: %) | | Initial | 3 months | 6 months |
| Example | 3 (Cysteine) | 0.3 | 0.8 | 1.9 |
| | 4 (Cysteine) | 0.4 | 0.8 | 1.4 |
| | 8 (Thioglycerol) | 0.2 | 0.4 | 0.6 |
| | 10 (Thioglycerol) | 0.2 | 0.2 | 0.3 |
| | 16 (N-acetylcysteine) | 0.3 | 0.6 | 1.5 |
| Comparative | 1 (None) | 0.1 | 12.9 | 28.9 |
| Example | | | | |

In Examples 3 and 4 in which cysteine was formulated, Examples 7 and 9 in which thioglycerol was formulated, and Example 13 in which N-acetylcysteine was formulated with an active ingredient of the present invention, the active ingredient was very effectively inhibited from being converted into the N-oxopyridine compound even in liquid eye drops, thereby showing excellent stability even in a long-term stability test for more than about six months.

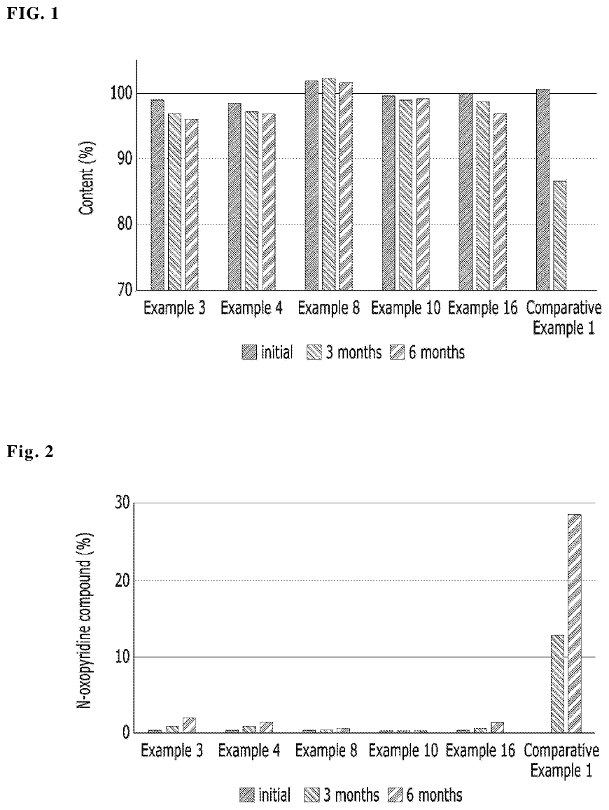

In contrast, in the case of Comparative Example 1, which does not contain an antioxidant, a content of the active ingredient decreased to 70% or less within about six months of storage after preparation, and 20.0% or more of the N-oxopyridine compound was generated. (FIGS. 1 and 2)

In other words, the eye drop composition according to the present invention may maintain excellent stability even under the long-term storage conditions (about 25° C./about 60% RH) for about one month or more, about two months or more, about three months or more, about four months or more, about five months or more, and about six months or more. In addition, the eye drop composition according to the present invention may maintain the occurrence of the N-oxopyridine compound in an amount of less than about 3% to ensure stability due to long-term storage, and thus can exhibit an excellent therapeutic effect for a long period of time and minimize side effects due to a decline in stability, thereby securing the safety of the drug.

A content range of a main ingredient of eye drops, which is generally acceptable in the industry, is 90 to 110%, and a content range of the N-oxopyridine compound, which has been confirmed to be safe in the composition of the present invention, is less than about 3%. Thus, it was confirmed that the active ingredient of the present invention needs to be formulated together with an antioxidant substance so that the active ingredient may specially secure long-term stability and may be developed into a drug medicine as an eye drop.

### Test Example 2. Evaluation of Efficacy of Eye Drop Composition Using Mouse CNV Model

In order to confirm the inhibitory effect of the eye drop composition of the present invention (Example 7) on choroidal neovascularization (CNV), the efficacy was evaluated with the mouse CNV model by selecting the composition of the present invention as a test material, a placebo material as a negative control material (G1), and Eylea®, which is a commercially available injection, as a positive control material (G2).

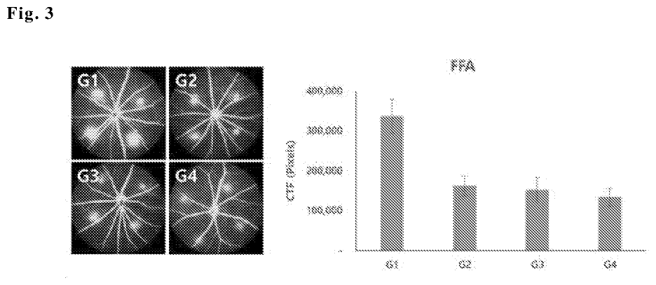

The negative control material (G1) and test materials (G3 and G4) were instilled at a dose of 5 uL once from the day after CNV induction, and the positive control material (G2) was directly injected once into a vitreous body at a dose of 1 uL (20 ug/uL, maximum dosage for the mouse) by using a 36 G syringe on the day after CNV induction. The negative control material was administered three times a day. With regard to the test material, efficacy was compared and evaluated according to the number of daily administrations in such a way that the groups were divided into an administration group of three times (G3) and an administration group of six times (G4). Specifically, on the 12th day after CNV induction, the experimental animal was subjected to general anesthesia and to intraperitoneal injection of a fluorescent contrast agent. Then, anesthetic eye drop was applied into the eyeball of the experimental animal to induce additional local anesthesia. Mydriatic was applied to induce mydriasis and perform a retinal image evaluation. Thus, the results are shown in FIG. 3.

As a result of the test, the compositions (G3, G4) of the present invention showed an effect equal to or higher than that of the group dosed with Eylea® (positive control group, G2), which is directly injected into the vitreous body. (FIG. 3) This means that the eye drop composition of the present invention with secured long-term stability and safety may allow the active ingredient to reach the posterior segment of the eyeball simply through instillation rather than a direct intraocular injection method, thereby exhibiting excellent efficacy in preventing or treating eye diseases.

1. An eye drop for preventing or treating eye diseases, comprising:
3-phenyl-4-propyl-1-(pyridin-2-yl)-1H-pyrazol-5-ol represented by formula 1 below or pharmaceutically acceptable salts thereof as an active ingredient; and N-oxopyridine compound represented by formula 2 below in an amount of less than about 3%:

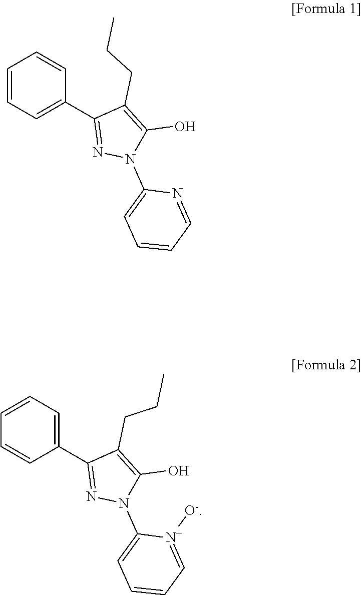

2. The eye drop of claim 1, further comprising an antioxidant substance.

3. An eye drop for preventing or treating eye diseases, comprising:
3-phenyl-4-propyl-1-(pyridin-2-yl)-1H-pyrazol-5-ol represented by formula 1 below or pharmaceutically acceptable salts thereof as an active ingredient; and
at least one antioxidant substance selected from cysteine, N-acetyl cysteine and monothioglycerol:

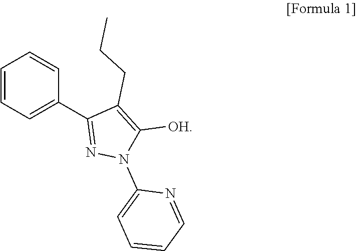 [SMILES: CCCc1c(-c2ccccc2)nn(-c2ccccn2)c1O]

4. The eye drop of claim 3, wherein the antioxidant substance is
monothioglycerol

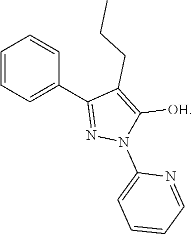 [SMILES: CCCc1c(-c2ccccc2)nn(-c2ccccn2)c1O]

5. The eye drop of claim 3, wherein the pharmaceutically acceptable salt is hydrochloride.

6. The eye drop of claim 3, comprising the antioxidant substance in an amount of about 0.1 wt % to about 5.0 wt % based on total weight of the eye drop.

7. The eye drop of claim 3, further comprising N-oxopyridine compound represented by formula 2 below in an amount of less than about 3%:

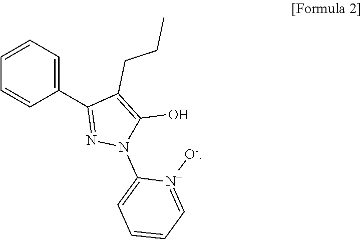

8. The eye drop of claim 7, wherein the eye drop comprises the N-oxopyridine compound represented by above formula 2 in an amount of less than about 3%, even after storage for about six months under a condition of about 25° C. temperature and about 60% relative humidity.

9. (canceled)

10. (canceled)

11. A method for preventing or treating eye diseases, the method comprising; administering an eye drop containing 3-phenyl-4-propyl-1-(pyridin-2-yl)-1H-pyrazol-5-ol represented by formula 1 below or a pharmaceutically acceptable salt thereof as an active ingredient; and N-oxopyridine compound represented by formula 2 below in an amount of less than about 3%, to a subject in need thereof:

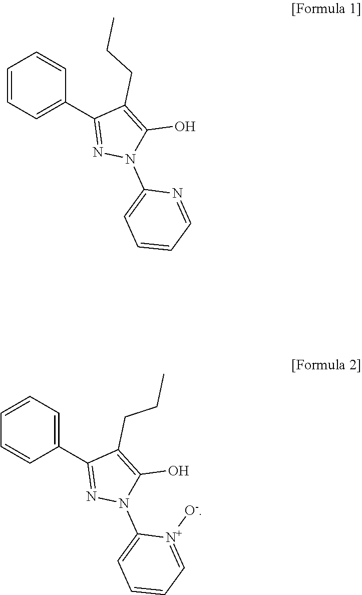

12. (canceled)

13. (canceled)

14. The eye drop of claim 1, further comprising at least one additive selected from the group consisting of solubilizing aids, surfactants, clathrate, and mixtures thereof.

15. The eye drop of claim 1, wherein the pharmaceutically acceptable salt is hydrochloride.

16. The eye drop of claim 2, wherein the antioxidant substance is selected from the group consisting of cysteine, N-acetylcysteine, and monothioglycerol.

17. The eye drop of claim 2, comprising the antioxidant substance in an amount of about 0.1 wt % to about 5.0 wt % based on total weight of the eye drop.

18. The method of claim 11, wherein the pharmaceutically acceptable salt is hydrochloride.

19. The method of claim 11, wherein the eye drop further comprises at least one antioxidant substance selected from group consisting of cysteine, N-acetylcysteine, and monothioglycerol.

20. The method of claim 19, wherein the antioxidant substance is monothioglycerol.

21. The method of claim 11, wherein the eye disease is a posterior segment eye disease.

22. The method of claim 21, wherein the posterior segment eye disease is at least one selected from the group consisting of diabetic retinopathy (DR), diabetic macular edema, age-related macular degeneration, acute macular neuroretinopathy, retinopathy of prematurity (ROP), polypoidal choroidal vasculopathy, ischemic proliferative retinopathy, retinitis pigmentosa, cone dystrophy, Behcet's disease, retinal disorders, proliferative vitreoretinopathy (PVR), retinal artery occlusion, retinal vein occlusion, retinitis, uveitis, Leber's hereditary optic neuropathy, retinal detachment, retinal pigment epithelial detachment, neovascular glaucoma, retinal neovascularization and choroidal neovascularization (CNV), trauma of the posterior segment, radiation retinopathy, epiretinal membrane disorder, branch retinal vein occlusion, anterior ischemic optic nerve disorder, non-retinopathy diabetic retinal dysfunction, and glaucoma.
